(19) United States
(12) Patent Application Publication
(10) Pub. No.: US 2008/0194474 A1
(43) Pub. Date: Aug. 14, 2008

(54) Methods for the Treatment of Autoimmune Diseases

(75) Inventor: Suzanne T. Ildstad, Prospect, KY (US)

Correspondence Address: FISH & RICHARDSON P.C., PO BOX 1022, MINNEAPOLIS, MN, 55440-1022, US

(73) Assignee: UNIVERSITY OF LOUISVILLE RESEARCH FOUNDATION, INC., Louisville, KY (US)

(21) Appl. No.: 11/569,116

(22) PCT Filed: May 13, 2005
(86) PCT No.: PCT/US05/16850

§ 371 Date: Apr. 1, 2008

Related U.S. Application Data
(60) Provisional application No. 60/571,206, filed on May 14, 2004.

(51) Int. Cl. A61K 38/18; C12Q 1/02; A61P 37/00

(52) U.S. Cl. 514/12; 435/29

(57) ABSTRACT

The invention provides for methods for causing myeloid precursor cells in bone marrow to differentiate into dendritic cells, methods of screening for compounds that relieve a block in the development of mature myeloid progeny and/or that increase the population of HSA⁺/Ly6C⁺ cells in bone marrow, and methods of preventing or delaying an autoimmune disease such as diabetes.

### CROSS REFERENCE TO RELATED APPLICATIONS

This application claims priority under 35 U.S.C. § 119(e) of U.S. Application No. 60/571,206, filed May 14, 2004, which is incorporated by reference herein.

### FEDERALLY SPONSORED RESEARCH OR DEVELOPMENT

The U.S. Government may have certain rights in this invention pursuant to Grant No. AI45486 awarded by the NIH and Grant No. HL063442 awarded by NHLBI.

### TECHNICAL FIELD

This invention relates to improving engraftment of tissue, and more particularly to methods of increasing the population of HSA⁺/Ly6C⁺ cells.

### BACKGROUND

Bone marrow transplantation (BMT) has the potential to treat a number of genetic disorders, including hemoglobinopathies (sickle cell disease, thalassemia), soluble enzyme deficiencies, and autoimmune disorders. The morbidity and mortality associated with transplantation of unmodified marrow has prevented the widespread application of this approach. Conventional T cell depletion prevents graft versus host disease but is associated with an unacceptably high rate of graft failure. A better understanding of the biology of engraftment of HSC will allow approaches to graft engineering to optimize engraftment and avoid the risks associated with BMT.

### SUMMARY

The invention provides for methods for causing myeloid precursor cells in bone marrow to differentiate into dendritic cells, methods of screening for compounds that relieve a block in the development of mature myeloid progeny and/or that increase the population of HSA⁺/Ly6C⁺ cells in bone marrow, and methods of preventing or delaying an autoimmune disease such as diabetes.

In one aspect, the invention provides methods of increasing the population of HSA⁺/Ly6C⁺ cells in bone marrow. The invention also provides for methods for causing myeloid precursor cells in bone marrow to differentiate into dendritic cells. Such a method generally includes contacting the bone marrow with an Fms-like tyrosine kinase 3 ligand (Flt3-L). Typically, the contacting step results in an increase in the HSA⁺/Ly6C⁺ cells in the bone marrow and/or causes the myeloid precursor cells in the bone marrow to differentiate into dendritic cells.

In certain embodiments, the bone marrow is donor marrow. Donor bone marrow can be in a donor, or in culture. In other embodiments, the bone marrow is chimeric bone marrow in a recipient. In some embodiments, an increase in HAS+/Ly6C+ cells can be determined by marker-specific flow cytometry. Representative Flt3-L polypeptides include a mouse Flt3-L polypeptide and a human Flt3-L polypeptide.

In another aspect, the invention provides for methods of screening for compounds that increase the population of HSA⁺/Ly6C⁺ cells in bone marrow. The invention also provides for methods of screening for compounds that relieve a block in the development of mature myeloid progeny. Such methods generally include contacting the bone marrow with a test compound, and detecting the presence or amount of HSA⁺/Ly6C⁺ cells in the presence of the test compound. Typically, an increased population of HSA⁺/Ly6C⁺ cells in the bone marrow compared to the population of HSA⁺/Ly6C⁺ cells in bone marrow not contacted with the test compound is indicative of a compound that relieves a block in the development of mature myeloid progeny and/or increases the population of HSA⁺/Ly6C⁺ in bone marrow.

In certain embodiment, the bone marrow is mouse bone marrow (e.g., bone marrow from a NOD mouse). In other embodiments, the bone marrow is human bone marrow (e.g., bone marrow from a diabetic). Representative autoimmune diseases include, without limitation, type I diabetes, rheumatoid arthritis, systemic lupus erythematosus, multiple sclerosis, psoriasis, scleroderma, inflammatory bowel diseases, and myasthenia gravis. Representative test compounds include, without limitation, an oligonucleotide, a peptide, a chemical compound, a mixture of chemical compounds, a bacterial extract, a plant extract, a fungal extract, or an animal extract. Typically, the block in the development of mature myeloid progeny is the results of an autoimmune disease.

In another aspect, the invention provides for methods of preventing or delaying an autoimmune disease (e.g., diabetes). Such methods generally include administering an effective amount of an Flt3-L polypeptide to the individual. Typically, the administering step prevents or delays the autoimmune disease. Such methods can further comprise identifying an individual at risk for developing an autoimmune disease (e.g., diabetes). In certain embodiment, the Flt3-L can be administered to the individual subcutaneously, orally, intramuscularly, or intravenously. As indicated above, representative autoimmune diseases include type I diabetes, rheumatoid arthritis, systemic lupus erythematosus, multiple sclerosis, psoriasis, scleroderma, inflammatory bowel diseases, and myasthenia gravis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 shows Ly6C and AA4.1 expression on HSA⁺ BMCs. BMCs from naïve BALB/c, B10.BR, NOD and NOR mice were stained and analyzed for the expression of HSA and Ly6C or AA4.1. Quadrant markers were based on individual isotype control staining. (A) Representative dot-plots from each of the strains tested are shown. (B) and (C) show data from individual animals (diamonds) and the means (lines) from one experiment. (B) compares the percentage of HSA⁺/Ly6C⁺ in the total BMC all strains tested, while (C) compares NOD and NOR BMC on a 10-fold lower scale than that of (B). (D) Representative HSA/AA4.1 plots from each of the strains tested are shown, and (E) shows results from 3 individual animals (diamonds) and the means (lines) from one experiment.

FIG. 2 shows that the HSA⁺/Ly6C⁺ population is restored in the bone marrow of fully allogeneic and mixed B10.BR→NOD chimeras. (A) Percent HSA⁺/Ly6C⁺ BMC from naïve B10.BR and NOD mice and from fully allogeneic NOD chimeras is shown (n=5 animals/group). (B) BMC from five B10.BR NOD mixed allogeneic chimeras (A-E) were analyzed for HSA/Ly6C co-expression. Percent donor chimerism refers to the level of donor-derived cells in the PBL at the time of analysis.

FIG. 3 shows that NOD BMC acquire an HSA⁺/Ly6C⁺ phenotype after in vitro culture with FL. BMC from NOD, B10.BR and C57BL/10 mice were cultured in the absence (A) and presence of FL (B) for up to 10 days. Cells harvested at the indicated time points were analyzed for HSA⁺/Ly6C⁺ co-expression. One of four experiments is shown. (C) FL-cultured cells from NOD, B10.BR and C57BL/10 mice were harvested at the indicated times and the HSA⁺/Ly6C⁺ events were analyzed for mDC as defined by CD11c and CD11b co-expression. Data are from one of two experiments.

FIG. 4 shows that in vivo treatment of NOD mice with FL delays progression of diabetes and significantly decreases insulitis at 14 weeks. (A) NOD mice were given saline (n=16) or FL (n=16) and monitored for diabetes. (B) Pancreata from 7 week NOD mice and 14 week NOD mice that had received either saline or FL starting at 9-weeks of age were scored for insulitis (Li et al., 1996, J. Immunol., 156:380-388). Data are from two combined experiments, between 120 and 300 individual islets were scored in a blinded fashion per pancreas Individual numbers for data shown are: 7 week, no FL n=3; 14 week, no FL n=5; 14 week+FL n=6. (C) Total BMC from age-matched untreated and FL-treated (+FL) NOD mice were stained for HSA and Ly6C immediately following therapy. Data are from individual mice (⋄) and the mean (—) from one experiment. P=<0.01. (D) The number of mDC(CD11c⁺/CD11b⁺/B220⁻) and pDC (CD11c⁺/CD11b⁻/B220⁺) per μl of PB was determined in NOD mice given either saline (no FL, n=6) or FL (+FL, n=6) daily for 10 days. Data presented are averages±standard deviation from six individual mice from two experiments. (E) The number of CD4⁺ CD25⁺ cells obtained from the panLN in untreated (open bar) and FL-treated NOD (closed bar) mice. Data are the average of three individual mice from one of two experiments.

Like reference symbols in the various drawings indicate like elements.

### DETAILED DESCRIPTION

This disclosure demonstrates that mixed chimerism supplies missing factors that correct for a defect in the differentiation of myeloid cells, and demonstrates that this defect in NOD mice is not due to an inability to produce myeloid precursor cells, but rather to a lack of some factor or cellular interaction that instructs precursor cells to this lineage. This disclosure also demonstrates that treatment of NOD mice with Fms-like tyrosine kinase 3 ligand (Flt3-L) restored the production of myeloid progenitor cells that co-express Ly6C and heat stable antigen (HSA) (referred to herein as HSA⁺/Ly6C⁺ cells) and increased the number of dendritic cells. Importantly, insulitis and diabetes progression were both significantly delayed in the Flt3-L-treated NOD mice. This disclosure provides in vivo evidence that peripheral tolerance mediated by myeloid-derived dendritic cells is critical to the control of autoimmune diseases.

Based on the disclosure herein, the invention provides for methods for causing myeloid precursor cells in bone marrow to differentiate into dendritic cells, methods of screening for compounds that relieve a block in the development of mature myeloid progeny and/or that increase the population of HSA⁺/Ly6C⁺ cells in bone marrow, and methods of preventing or delaying an autoimmune disease such as diabetes.

### Methods of Increasing the Population of HSA⁺/Ly6C⁺ Cells

The invention provides for methods of increasing the population (i.e., number) of HSA⁺/Ly6C⁺ cells in bone marrow. The number of HSA⁺/Ly6C⁺ cells can be increased by contacting bone marrow with an Flt3-L polypeptide. This disclosure demonstrates that in the presence of Flt3-L, myeloid precursor cells are able to differentiate into dendritic cells. Thus, the number of HSA⁺/Ly6C⁺ cells is increased.

The population of HSA⁺/Ly6C⁺ cells in bone marrow can be increased at different times during a transplantation process. For example, the population of HSA⁺/Ly6C⁺ cells can be increased in the donor bone marrow prior to harvest. Alternatively or in addition, bone marrow can be harvested from a donor and the population of HSA⁺/Ly6C⁺ cells can be increased in culture prior to transplanting the bone marrow into a recipient. In addition to increasing the population of HSA⁺/Ly6C⁺ cells in donor bone marrow, the population of HSA⁺/Ly6C⁺ cells can be increased upon transplantation into the recipient.

The presence or amount of HSA⁺/Ly6C⁺ cells can be determined using marker-specific flow cytometry as described herein. In addition, a microarray can be used to determine the presence or absence of HSA⁺ and Ly6C⁺ markers in cells using standard protocols. Data from a microarray can be acquired with, for example, a GeneChip Scanner 3000 (Affymetrix), and analyzed, for example, using GeneSpring software (Silicon Genetics). Conventional methods to detect nucleic acids encoding HSA and Ly6C markers or the HSA and Ly6C polypeptides themselves also can be used to detect cells exhibiting the HSA⁺/Ly6C⁺ markers. See, for example, Sambrook et al. (2001, Molecular Cloning: A Laboratory Manual, 3rd Ed., Cold Spring Harbor Laboratory Press, Cold Spring Harbor, N.Y.).

As demonstrated herein, increasing the population of HSA⁺/Ly6C⁺ cells can delay or prevent an autoimmune disease such as type I diabetes, rheumatoid arthritis, systemic lupus erythematosus, multiple sclerosis, psoriasis, scleroderma, inflammatory bowel diseases, myasthenia gravis, autoimmune hemolytic anemia, Goodpasture's syndrome, Hashimoto's thyroiditis, pernicious anemia, Addison's disease, dermatomyositis, Sjogren's syndrome, Reiter's syndrome, Grave's disease, or Wegener's granulomatosis. The population of HSA⁺/Ly6C⁺ cells can be increased in an individual already experiencing an autoimmune disease, or the population of HSA⁺/Ly6C⁺ cells can be increased prophylactically in an individual at risk for developing an autoimmune disease. Individual's can be identified as at risk for developing an autoimmune disease using methods known in the art as well as a family history of autoimmune disease. Numerous imaging and laboratory tests such as, for example, a complete blood count (CBC) and an erythrocyte sedimentation rate (ESR or sed rate) as well as those that detect antinuclear antibodies (ANA), complement, and/or creatinine can be used to diagnose or evaluate an autoimmune disease.

### Flt3-L Polypeptides

Fms-like tyrosine kinase 3 ligand (Flt3-L) is a hematopoietic growth factor that enhances the survival and expansion of bone marrow cells. In contrast to granulocyte-macrophase colony-stimulating factor (GM-CSF), Flt3-L preferentially induces the expansion of type 1 T cells. Flt3-L also regulates apoptosis through AKT-dependent inactivation of a transcription factor, FoxO3. For a review of Flt3-L, see, for example, Antonysamy & Thomson, 2000, Cytokine, 12:87-100; and Drexler & Quentmeier, 2004, Growth Factors, 22:71-3. See, also, WO 94/28391, WO 94/26891, and U.S. Pat. No. 5,554,512.

The nucleic acids encoding a Flt3-L have been cloned from human and mouse in addition to numerous other species. The sequence of mouse Flt3-L can be found, for example, at GenBank Accession No. NP_{—}038548. The sequence of the human Flt-3 can be found, for example, at GenBank Accession No. NP_{—}001450. See, also, GenBank Accession Nos. NM_{—}010229, NM_{—}013520, NM_{—}004119, and NM_{—}001459 for additional Flt3-L sequences. Polypeptides that are in the same family as Flt3-L can also be used in the methods disclosed herein, provided such polypeptides increase the population of HSA⁺/Ly6C⁺ cells. For example, flk-2 is a tyrosine kinase receptor and has an amino acid sequence that is very similar to that of Flt3-L. See Matthews et al., 1991, Cell, 65:1143-52.

As discussed above, bone marrow cells can be contacted in culture with an Flt3-L polypeptide, or an Flt3-L polypeptide can be administered to a donor or a recipient. An Flt3-L polypeptide can be administered to an individual in a number of ways including, but not limited to, subcutaneously, orally, intramuscularly, and intravenously. For administration to a donor or recipient, an Flt3-L composition generally includes a pharmaceutically acceptable carrier. As used herein, “pharmaceutically acceptable carrier” is intended to include any and all solvents, dispersion media, coatings, antibacterial and anti-fungal agents, isotonic and absorption delaying agents, and the like, compatible with pharmaceutical administration. The use of such media and agents for pharmaceutically active substances is well known in the art. The particular pharmaceutically acceptable carrier is formulated to be compatible with its intended route of administration.

Methods for Screening for Compounds that Increase the Population of HSA⁺/Ly6C⁺ Cells

This disclosure demonstrates that increasing or restoring the HSA⁺/Ly6C⁺ cell population in bone marrow can delay the progression of or prevent an autoimmune disease. Therefore, the disclosure herein provides for methods of screening for compounds that relieve a block in the development of mature myeloid progeny. Such a block in the development of mature myeloid progeny can be the result of an autoimmune disease. As described herein, relieving a block in the development of mature myeloid progeny results in an increase in the population of HSA⁺/Ly6C⁺ cell.

Methods of screening for compounds that increase the population of HSA⁺/Ly6C⁺ cells include contacting bone marrow with a test compound, and detecting the presence or amount of HSA⁺/Ly6C⁺ cells. Compounds that can be screened for the ability to relieve a block in the development of mature myeloid progeny and increase the population of HSA⁺/Ly6C⁺ cell can be, for example, without limitation, a biological macromolecule, such as an oligonucleotide or a peptide, a chemical compound, a mixture of chemical compounds, or an extract isolated from bacterial, plant, fungal or animal matter. A compound is identified as being able to relieve the block in development of mature myeloid progeny and/or increase the population of HSA⁺/Ly6C⁺ cells if the population of HSA⁺/Ly6C⁺ cells in the bone marrow that was exposed to the compound is increased compared to the population of HSA⁺/Ly6C⁺ cells in a control sample (i.e., bone marrow that was not contacted with the test compound).

The bone marrow used to test compounds can be mouse bone marrow (e.g., from a NOD mouse), or human bone marrow. The human bone marrow can be from a diabetic, or from an individual that suffers from another autoimmune disease such as, without limitation, type I diabetes, rheumatoid arthritis, systemic lupus erythematosus, multiple sclerosis, scleroderma, inflammatory bowel diseases, or myasthenia gravis. In addition, there are animal models of several autoimmune diseases can be used to screen for compounds that relieve the block in development of mature myeloid progeny and/or increase the population of HSA⁺/Ly6C⁺ cells. For example, NOD mice are a well characterized model for diabetes, and have well-characterized abnormalities that affect antigen presentation. NOD mice also exhibit defective production of myeloid progeny in bone marrow cells, most notably, resulting in impaired responses to cytokines including IL-3, IL-5, and granulocyte macrophage colony-stimulating factor (GM-CSF). For information on several different animal models of autoimmune diseases, see, for example, U.S. Pat. No. 6,828,472; and Zandman-Goddard & Shoenfeld, 2005, Lupus, 14:12-16; Lam-Tse et al., 2002, Springer Semin. Immunopathol., 24:297-321; Infante & Kraig, 1999, Int. Rev. Immunol., 18:83-109; and Morel, 2004, PLoS Biol., 2:e241.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the invention described in the claims.

### EXAMPLES

### Example 1

### Materials and Methods

Animals. Four- to six-week-old female NOD/Lt mice were purchased from Taconic Laboratories, (Germantown, N.Y.). Four to five-week-old female BALB/cByJ, NOR/Lt, B10.BR/SgSnJ and C57BL/10SnJ mice were purchased from the Jackson Laboratory (Bar Harbor, Mass.). Animals were housed in a barrier animal facility at the Institute for Cellular Therapeutics, University of Louisville, and cared for according to National Institutes of Health animal care guidelines.

Antibodies. All monoclonal antibodies (mAb) used in this study were purchased from BD/Pharmingen (San Diego, Calif.) and included mAb against HSA-PE, AA4.1-FITC, Ly6C-biotin or -FITC, H-2K^{d}-FITC, H-2K^{k}-PE, CD11c-APC or -FITC, CD11b-APC or -FITC, B220-PerCP, CD80-FITC, and CD86-FITC.

Chimera preparation. BMCs were harvested and resuspended to 100×10⁶ cells/ml in chimera media (CM, Medium 199, [GIBCO/BRL, Grand Island, N.Y.] with 50 μg/ml gentamicin [GIBCO]). Fully allogeneic NOD chimeras were prepared by irradiating NOD mice (H2^{g7}) with 1000 cGy TBI (Gamma-cell 40, Nordion, Ontario, Canada) and administering 30×10⁶ B10.BR BMC in CM as previously described (Li et al., 1996, J. Immunol., 156:380-388). For mixed allogeneic chimeras, NOD mice were given either 750 cGy TBI 4-6 hours prior to infusion with untreated 30×10⁶ B10.BR BMCs or 950 cGy TBI plus 40×10⁶ T cell-depleted B10.BR BMCs.

Assessment of Chimerism. Recipients were characterized for allogeneic engraftment using two-color-flow cytometry thirty days post-transplantation as previously described (Xu et al., 2004, J. Immunol., 172:1463-1471). Briefly, whole blood was collected and 100 μl aliquots were stained with anti-H-2 K^{d}-FITC and anti-H-2K^{k} PE for 30 minutes and either analyzed fresh or fixed in a 1% formaldehyde (Polysciences, Warrington, Pa.) until analysis. Data were acquired on a FACSCalibur flow cytometer (Becton Dickinson, San Diego, Calif.) and analyzed using CellQuest software (Becton Dickinson).

In vitro culture of BMCs. BMCs from NOD, B10.BR and C57BL/10 mice were harvested and placed in culture at 33° C. at 5% CO₂. Cells were cultured at 1×10⁶ cells/ml in long-term BMC media (LT-BMC; IMDM [GIBCO] supplemented with 20% horse serum [GIBCO], 10⁻⁴ M β-mercaptoethanol [Sigma], 10⁻⁵ M hydrocortisone [Sigma], 100 U/ml penicillin, 100 mg/ml streptomycin [GIBCO], 2 mM L-glutamine [GIBCO]) in the absence and presence of the following cytokines used singly or in combination: GM-CSF (10³μ/mil) (Genzyme, Cambridge, Mass.), SCF (1.2 U/ml) (Genzyme) and FL (10 ng/ml) (generously provided by Amgen, Thousand Oaks, Calif.). Cells were harvested at selected time points and stained for the co-expression of HSA and Ly6C as well as DC subsets: mDC(CD11c⁺/CD11b⁺/B220⁻) and pDC(CD11c⁺/CD11b⁻/B220⁺).

In vivo FL treatment of NOD mice. Pre-diabetic (7-9 week old) female NOD mice were treated daily for 10 days with 10 μg of FL subcutaneously. On day 11, peripheral blood and collagenase-digested spleen and pancreatic lymph nodes were analyzed for the presence of DC, T cells and NK cells and compared to untreated age-matched control NOD mice. Other groups were treated with the 10-day course of FL (10 μg/day) or saline and followed for diabetes progression or insulitis. Progression of diabetes was monitored using urine glucose testing using Chemstrip uGK test strips (Roche, Indianapolis, Ind.). From the fixed pancreatic samples, 0.7 μm sections were obtained from control and FL-treated mice, stained with hematoxylin and eosin and assessed for insulitis as previously described (Kagi et al., 1999, J. Immunol., 162:4598-4605). A minimum of 120 islets were scored per sample on a blinded basis.

Statistical analysis. Diabetes progression curves of the FL-treated or saline controls were compared using SPSS for Windows 11.0.1 statistical software package. A Cox-regression analysis was performed with the age at diabetes set as the timed event. Each treatment group consisted of 16 mice followed until at least 40 weeks of age. Log-rank results indicate that the P value comparison between the two curves was 0.0084 or P<0.01.

### Example 2

### Results

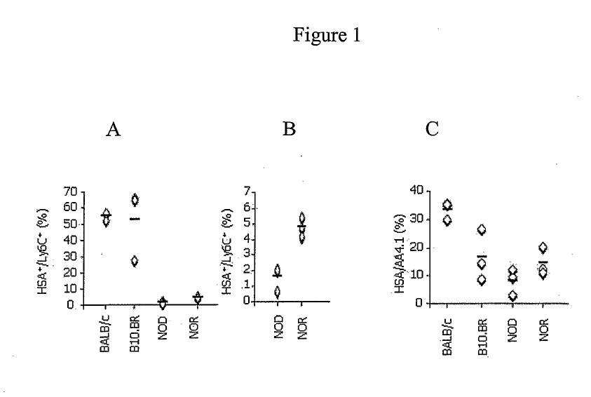

BMC from NOD mice express decreased numbers of HSA⁺/Ly6C⁺ cells compared to B10.BR, BALB/c or NOR mice. BMCs from age-matched NOD B10.BR and BALB/c mice were analyzed for the co-expression of Ly6C or AA4.1 with HSA. As previously described, NOD BMC lack a distinct population of HSA⁺/Ly6C⁺ cells. The HSA⁺/Ly6C⁺ population represents 1.6%±0.8% of the NOD BMC in contrast with 52.8%±22.0% of the B10.BR(P<0.05) and 56.8%±2.5% BALB/c (P<0.001) BMC. Although the HSA⁺/Ly6C⁺ population in BMC from congenic diabetes-resistant NOR mice was significantly lower compared to either BALB/c or B10.BR strains (P<0.05 each), this value was nevertheless 3-fold greater than in NOD BMC (FIG. 1B; 4.8±0.6%; P<0.01). Furthermore, when NOD BMC were stained for the mDC markers CD11b and CD11c, a deficiency in the CD11c⁺ population was noted. The NOD CD11c⁺ cells stain only dimly compared to controls, indicative of an immature mDC population. Notably, the more bright population of mature mDC is lacking.

In contrast, no difference was detected in the HSA⁺/AA4.1⁺ cell population between NOD and B10.BR mice (P>0.05; FIG. 1C). It was suspected that the difference between the results disclosed herein and that from other groups was due to strain differences. When BALB/c BMC were analyzed, it was determined that BALB/c BMC have significantly more HSA⁺/AA4.1⁺ cells compared to BMC from B10.BR, NOR or NOD (P<0.05 each). Since the differences in the HSA⁺/AA4.1⁺ cell population did not correlate with the diabetic phenotype of the NOD mouse, the remaining studies focused on the myeloid HSA⁺/Ly6C⁺ cell population.

The bone marrow HSA⁺/Ly6C⁺ population in B10.BR and C57BL/10 mice is myeloid. In order to determine the basis for the cellular deficiency in the HSA⁺/Ly6C⁺ population in NOD, BMCs were harvested from NOD, B10.BR and C57BL/10 mice and analyzed for other lineage markers. When the HSA⁺/Ly6C⁺ cells were gated and analyzed for the expression of CD11b and CD11c, nearly 100% of the HSA⁺/Ly6C⁺ population was CD11b⁺. Of these cells, approximately 30% also expressed CD11c, a phenotype typical of mDC.

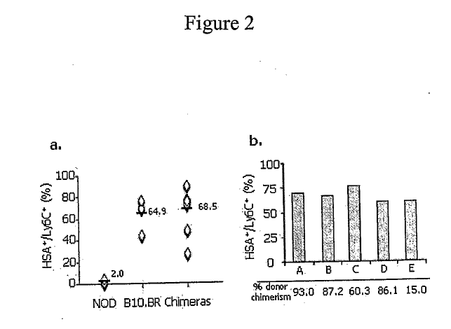

Fully chimeric NOD mice express the HSA⁺/Ly6C⁺ cell population similar to donor strain levels. To examine whether chimerism corrected the myeloid defect in the NOD marrow, B10.BR→NOD chimeras were prepared and one month after transplantation, the recipients were typed for donor chimerism. All mice (n=9) exhibited>99% donor B10.BR-derived cells in peripheral blood (PB). BMCs from chimeras were harvested and examined for the co-expression of HSA and Ly6C. The HSA⁺/Ly6C⁺ cell population in the chimeric NOD bone marrow (68.5%±19.0%) was significantly greater compared to naïve NOD mice (FIG. 2A; 2.0%±1.2%; P<0.05), and was similar to the HSA⁺/Ly6C⁺ cell population in naïve B10.BR mice (64.9%±13.9%; P=0.35).

Myeloid bone marrow cell expression in B10.BR→NOD mixed chimeric mice is similar to donor bone marrow. Whether or not the restored expression was due solely to the donor cells or if NOD-derived cells contributed to the HSA⁺/Ly6C⁺ population was examined. Mixed B10.BR→NOD chimeras were prepared and typed for chimerism at one month. The percent B10.BR derived cells in PB of these mice at the time of analysis ranged from 15.0 to 93.0% (n=5). The HSA⁺/Ly6C⁺ cell population in the bone marrow from the mixed chimeras (FIG. 2B) was significantly increased over that of un-manipulated NOD mice (FIGS. 1 and 2A). Additionally, the proportion of donor chimerism did not correlate with the increase in the HSA⁺/Ly6C⁺ cell population. All chimeras had levels of HSA⁺/Ly6C⁺ comparable to B10.BR mice (FIGS. 1 and 2A).

The HSA⁺/Ly6C⁺ population was further analyzed for the relative contribution by NOD vs. B10.BR bone marrow cells using flow cytometry. A representative HSA/Ly6C stain of the total BMC population from a B10.BR→NOD mixed chimera was compared to controls. Both B10.BR-derived H2-K^{k+} cells and NOD-derived H2-K^{d+} cells from chimeric mice contributed to the HSA⁺/Ly6C⁺ population. These data indicate that the lack of the HSA⁺/Ly6C⁺ population in naïve NOD mice is not due to an intrinsic inability to produce myeloid precursors, but rather due to a lack of signal(s) that would instruct precursor cells to this lineage.

The level of expression of MHC class I antigens on the cells in the myeloid gate is relatively low, especially for the HSA⁺/Ly6C⁺ population. In order to assure that the cells were of both donor and recipient origin, the analysis included only those cells that stained the brightest for MHC class I. There was a distinct population of NOD-derived HSA⁺/Ly6C⁺ cells.

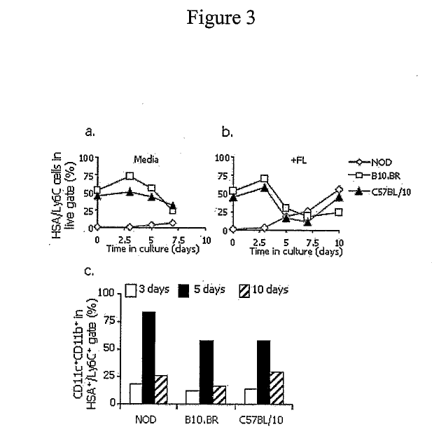

In vitro culture of NOD BMC results in the expression of an HSA⁺/Ly6C⁺ cell population. Whether or not NOD BMC could be induced to produce the HSA⁺/Ly6C⁺ population in vitro was examined. BMCs from NOD, B10.BR and C57BL/10 mice were harvested and placed in culture with the following hematopoietic growth factors used singly or in combination: GM-CSF, SCF, and FL. Interestingly, culture of NOD BMC in LT-BMC media alone resulted in a slight increase in the HSA⁺/Ly6C⁺ population, from 0.6% in fresh cells to 6.8% after 7 days in culture (FIG. 3A). The HSA⁺/Ly6C⁺ population decreased in the B10.BR and C57BL/10 cultures with time. The HSA⁺/Ly6C⁺ populations in the 7-day cultures was lower than in fresh BMCs, ranging from 52.8 to 24% and from 44.7 to 32%. However, the percentage of cells in these cultures was always higher than that in the NOD cultures (FIG. 3A). Viable cells were not obtained on day 10 when the cells were cultured in media alone; therefore, no day 10 results are shown.

Co-culture of NOD BMCs with SCF or GM-CSF did not result in a significant increase in the HSA⁺/Ly6C⁺ population compared to co-culturing with media alone. When FL was added to the BMC cultures, however, the HSA⁺/Ly6C⁺ population was significantly increased as early as day 5 (FIG. 3B). The percentage of HSA⁺/Ly6C⁺ cells in the culture continued to increase over time to levels similar to or greater than the control strains. The combination of FL, SCF and GM-CSF was not different from FL alone.

All of the HSA⁺/Ly6C⁺ cells are CD11b⁺, with many of the cells co-expressing CD11c. When NOD BMCs were cultured with FL, the percent CD11b⁺/CD11c⁺ mDC present within the HSA⁺/Ly6C⁺ cell population increased to a level similar to that in B10.BR and C57BL/10 cultures, with each culture reaching the highest percentage of mDC on day 5 (FIG. 3C). In addition, the HSA⁺/Ly6C⁺/CD11c⁺ population was also bright for MHC class II staining, indicative of mature mDC. Taken together, these data indicated that culture of NOD BMCs with FL was sufficient for differentiation of the myeloid precursor cells into mature mDC, confirming the in vivo observation that precursor cells are present in NOD marrow but need the proper signal for differentiation to occur.

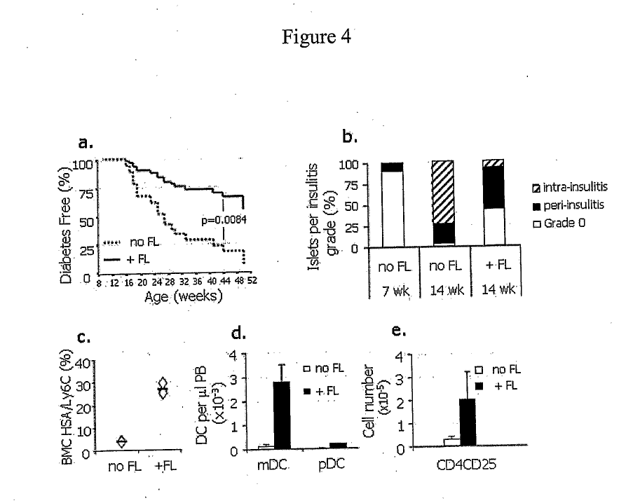

In vivo treatment of NOD mice with FL decreases insulitis and delays onset of diabetes. It was therefore hypothesized that treatment of NOD mice with FL in vivo would restore myeloid cell differentiation and lead to the generation of mature mDC that would help to control the peripheral autoimmune processes that lead to diabetes. To test this hypothesis, 7-9 week old pre-diabetic NOD mice were treated with either FL (10 μg/day) or saline for 10 consecutive days and monitored for diabetes progression. The first of the untreated NOD mice became diabetic at 16 weeks of age (FIG. 4A). At 40 weeks, 75% of untreated mice had developed diabetes. In the FL-treated group, disease progression was significantly delayed or prevented (P<0.01). The first conversion to diabetes in the FL-treated group did not occur until 24 weeks of age and 70% of the animals remained disease-free at 44 weeks.

Insulitis dramatically increases between the ages of 7 and 14 weeks in NOD mice (FIG. 4B). Strikingly, a significant decrease in insulitis was detected in the 14-week old NOD mice treated with a single 10-day course of FL starting at 9-weeks of age. The percentage of islets with no lymphocytic infiltration at 14 weeks was increased with FL treatment (4% untreated to 44.2% FL-treated). Although the islets exhibiting peri-insulitis increased more than two-fold in the FL-treated mice (22.8% vs. 47.8%, respectively), the percentage of islets exhibiting more aggressive intra-insulitis was significantly decreased compared to untreated age-matched controls (73.2% to 8.1%; P<0.05).

FL treatment of NOD mice increases HSA⁺/Ly6C⁺ cells in BM and mobilizes predominantly mDC. FL-treated NOD mice were evaluated for an increase in HSA⁺/Ly6C⁺ cells in the bone marrow and mobilization of mDC and pre-DC into PB. The HSA⁺/Ly6C⁺ population is indeed significantly increased in the NOD bone marrow following BMC FL treatment (26.8±2.2%) compared to untreated NOD BMCs (3.7±0.3%; P<0.01). Although both DC subsets were detected in the peripheral blood, the majority were of mDC phenotype (FIG. 4D), paralleling the in vitro data for culture of NOD BMC with FL. There was also a significant increase in both mDC and pre-DC in the spleen and panLN after treatment with FL as long as 5 weeks after treatment (Tables I and II, respectively). Notably, the mDC obtained from the panLN after FL treatment were significantly increased in number and had increased cell surface expression of both CD80 and CD86. However, CD80 and CD86 expression was not increased in the mDC found in the spleen (Tables I and II). Increased costimulatory molecule expression is indicative of mature mDC that have been shown to delay diabetes onset upon adoptive transfer into naïve NOD mice. Concomitant with the increase in mature mDC in the panLN is a six-fold increase in the numbers of CD4⁺/CD25⁺ T cells in the FL-treated NOD mice compared to untreated controls (P<0.05), indicative of generation of T regulatory cells (FIG. 4E).

TABLE I
Expansion of dendritic cell subsets in the spleen and their expression
of maturation markers after one 10-day course of FL in vivo
Average ± S.D.

| | | cell number (×10⁻⁶) | Fold increase | I-A⁺ (%) | CD80⁺ (%) | CD86⁺ (%) |
|---|---|---|---|---|---|---|
| mDC | no FL | 8.84 ± 0.8 | — | 81.9 ± 0.81 | 64.8 ± 2.8 | 41.7 ± 1.3 |
| | +FL | 114 ± 28.9 | 12.9 | 51.9 ± 1.04 | 66.7 ± 4.7 | 51.5 ± 5.9 |
| pDC | no FL | 4.96 ± 1.37 | — | 95.8 ± 0.98 | 34.8 ± 6.3 | 52.2 ± 2.4 |
| | +FL | 29.0 ± 5.27 | 5.8 | 91.0 ± 1.39 | 52.9 ± 1.4 | 27.2 ± 0.8 |

TABLE II
Expansion of dendritic cell subsets in the pancreatic lymph nodes and their
expression of maturation markers after one 10-day course of FL in vivo
Average ± S.D.

| | | cell number (×10⁻⁵) | Fold increase | I-A⁺ (%) | CD80⁺ (%) | CD86⁺ (%) |
|---|---|---|---|---|---|---|
| mDC | no FL | 0.01 ± 0.006 | — | 96.4 ± 1.1 | 37.0 ± 5.26 | 53.6 ± 5.1 |
| | +FL | 13.8 ± 14.2 | 1380 | 98.5 ± 1.3 | 77.6 ± 9.7 | 61.1 ± 13.8 |
| pDC | no FL | 0.12 ± 0.02 | — | 100 ± 0.0 | 33.9 ± 6.8 | 56.0 ± 6.7 |
| | +FL | 1.63 ± 1.05 | 13.6 | 99.0 ± 0.6 | 57.8 ± 8.8 | 34.2 ± 9.5 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of increasing the population of HSA⁺/Ly6C⁺ cells in bone marrow, comprising:
contacting said bone marrow with an Fms-like tyrosine kinase 3 ligand (Flt3-L),
wherein said contacting results in an increase in said HSA⁺/Ly6C⁺ cells in said bone marrow.

2. The method of claim 1, wherein said bone marrow is donor marrow.

3. The method of claim 2, wherein said donor bone marrow is in a donor.

4. The method of claim 2, wherein said donor bone marrow is in culture.

5. The method of claim 1, wherein said bone marrow is chimeric bone marrow in a recipient.

6. The method of claim 1, wherein an increase in HAS+/Ly6C+ cells is determined by marker-specific flow cytometry.

7. The method of claim 1, wherein said Flt3-L polypeptide is a mouse Flt3-L polypeptide or a human Flt3-L polypeptide.

8. A method for causing myeloid precursor cells in bone marrow to differentiate into dendritic cells, comprising:
contacting said bone marrow with an Flt3-L polypeptide,
wherein said contacting causes said myeloid precursor cells in said bone marrow to differentiate into dendritic cells.

9. A method of screening for compounds that increase the population of HSA⁺/Ly6C⁺ cells in bone marrow, comprising:
contacting said bone marrow with a test compound; and
detecting the presence or amount of HSA⁺/Ly6C⁺ cells in the presence of said test compound,
wherein an increased population of HSA⁺/Ly6C⁺ cells in said bone marrow compared to the population of HSA⁺/Ly6C⁺ cells in bone marrow not contacted with said test compound is indicative of a compound that increases the population of HSA⁺/Ly6C⁺ in bone marrow.

10. The method of claim 9, wherein said bone marrow is mouse bone marrow.

11. The method of claim 10, wherein said mouse is a NOD mouse.

12. The method of claim 9, wherein said bone marrow is human bone marrow.

13. The method of claim 12, wherein said human is diabetic.

14. The method of claim 12, wherein said human has an auto-immune disease selected from the group consisting of type I diabetes, rheumatoid arthritis, systemic lupus erythematosus, multiple sclerosis, psoriasis, scleroderma, inflammatory bowel diseases, and myasthenia gravis.

15. The method of claim 9, wherein said test compound is an oligonucleotide, a peptide, a chemical compound, a mixture of chemical compounds, a bacterial extract, a plant extract, a fungal extract, or an animal extract.

16. A method of screening for compounds that relieve a block in the development of mature myeloid progeny, comprising:
contacting bone marrow with a test compound; and
detecting the presence or amount of HSA⁺/Ly6C⁺ cells in the presence of said test compound,
wherein an increased population of HSA⁺/Ly6C⁺ cells in said bone marrow compared to the population of HSA⁺/Ly6C⁺ cells in bone marrow not contacted with said test compound is indicative of a compound that increases the population of HSA⁺/Ly6C⁺ in bone marrow.

17. The method of claim 16, wherein said block in the development of mature myeloid progeny is the results of an autoimmune disease.

18. A method of preventing or delaying diabetes in an individual, comprising:
administering an effective amount of an Flt3-L polypeptide to said individual,
wherein said administering results in an increase in said HSA⁺/Ly6C⁺ cells in said bone marrow.

19. The method of claim 18, further comprising identifying an individual at risk for developing diabetes.

20. The method of claim 18, wherein the Flt3-L is administered to said individual subcutaneously, orally, intramuscularly, or intravenously.

21. A method of preventing or delaying an autoimmune disease, comprising:
administering an effective amount of an Flt3-L polypeptide to said individual, wherein said administering prevents or delays said autoimmune disease.

22. The method of claim 21, wherein said autoimmune disease is selected from the group consisting of type I diabetes, rheumatoid arthritis, systemic lupus erythematosus, multiple sclerosis, psoriasis, scleroderma, inflammatory bowel diseases, and myasthenia gravis.
